SEQUENCE LISTING (8 sequences)

SEQ ID NO: 1 - DNA, 1374 bp, Eubacterium sabbureum DSM 3986
gtgaaaacaaaaaacaacattatatgtactattgcattgaaaggagacatatttatgaaagaattttttcccggcatatcacctgtaaagtttgagggcagagatagtaaaaatccacttagtttcaaatattatgatgccaaaagggtgataatgggcaaaacaatggaggaacatttatcatttgctatggcatggtggcataatctttgtgcctgtggtgtggatatgttcggacagggtactgtcgataaaagttttggtgaaagctccggtactatggagcatgcaagggctaaagtggatgcaggcattgaatttatgaaaaagcttggtataaagtattattgcttccatgatacggatattgtacctgaggatcaggaagatataaatgttaccaatgcacgtttggatgagattacagactatatcttagaaaaaacaaaggataccgatattaaatgtctttggacaacctgcaatatgttcagtaatccaagatttatgaacggtgcaggaagctcaaacagtgcagatgtattttgctttgcagcggcacaggcaaagaaaggtcttgaaaatgccgtaaaacttggagcaaagggatttgtattctggggaggcagagaaggttatgagacacttctaaatacagatatgaagcttgaagaggaaaatatagcaacactctttacaatgtgcagagattatggacgcagtataggctttatgggagatttttatattgagcctaagccgaaggagcctatgaagcatcagtatgattttgatgcggcaactgcaatcggttttttaagaaaatatggacttgataaagatttcaaactaaatattgaggcaaatcacgctacacttgcaggtcatacttttcagcatgagttaagagtatgtgcagtcaacggtatgatggggtcggtagatgccaatcaaggagatacattacttggatgggacactgatcaattccctacaaatgtctatgatactacattggctatgtatgaaatattaaaggcaggcggactccgtggaggtctgaactttgattcaaagaatcgcagaccaagtaatacagccgatgatatgttctatggctttatagcaggtatggacacatttgcacttggacttattaaggcggcggaaattatagaagacggaagaatagatgattttgttaaagaaagatatgcaagttataattcaggaataggtaagaagataagaaacagaaaagtgacactgatagagtgtgccgagtatgccgcaaagcttaaaaagcctgaactgccggaatcaggaagacaggaatatcttgagagcgtagtgaataatatattgttcggataa

SEQ ID NO: 2 - protein, 457 aa, Eubacterium sabbureum DSM 3986
MKTKNNIICTIALKGDIFMKEFFPGISPVKFEGRDSKNPLSFKYYDAKRVIMGKTMEEHLSFAMAWWHNLCACGVDMFGQGTVDKSFGESSGTMEHARAKVDAGIEFMKKLGIKYYCFHDTDIVPEDQEDINVTNARLDEITDYILEKTKDTDIKCLWTTCNMFSNPRFMNGAGSSNSADVFCFAAAQAKKGLENAVKLGAKGFVFWGGREGYETLLNTDMKLEEENIATLFTMCRDYGRSIGFMGDFYIEPKPKEPMKHQYDFDAATAIGFLRKYGLDKDFKLNIEANHATLAGHTFQHELRVCAVNGMMGSVDANQGDTLLGWDTDQFPTNVYDTTLAMYEILKAGGLRGGLNFDSKNRRPSNTADDMFYGFIAGMDTFALGLIKAAEIIEDGRIDDFVKERYASYNSGIGKKIRNRKVTLIECAEYAAKLKKPELPESGRQEYLESVVNNILFG

SEQ ID NO: 3 - protein, 440 aa, Escherichia coli
MQAYFDQLDRVRYEGSKSSNPLAFRHYNPDELVLGKRMEEHLRFAACYWHTFCWNGADMFGVGAFNRPWQQPGEALALAKRKADVAFEFFHKLHVPFYCFHDVDVSPEGASLKEYINNFAQMVDVLAGKQEESGVKLLWGTANCFTNPRYGAGAATNPDPEVFSWAATQVVTAMEATHKLGGENYVLWGGREGYETLLNTDLRQEREQLGRFMQMVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKIAARMIEDGELDKRIAQRYSGWNSELGQQILKGQMSLADLAKYAQEHHLSPVHQSGRQEQLENLVNHYLFDK

SEQ ID NO: 4 - protein, 438 aa, Clostridium phytofermentas
MKNYFPNVPEVKYEGPNSTNPFAFKYYDANKVVAGKTMKEHCRFALSWWHTLCAGGADPFGVTTMDRTYGNITDPMELAKAKVDAGFELMTKLGIEFFCFHDADIAPEGDTFEESKKNLFEIVDYIKEKMDQTGIKLLWGTANNFSHPRFMHGASTSCNADVFAYAAAKIKNALDATIKLGGKGYVFWGGREGYETLLNTDLGLELDNMARLMKMAVEYGRANGFDGDFYIEPKPKEPTKHQYDFDTATVLAFLRKYGLEKDFKMNIEANHATLAGHTFEHELAMARVNGAFGSVDANQGDPNLGWDTDQFPTDVHSATLAMLEVLKAGGFTNGGLNFDAKVRRGSFEFDDIAYGYIAGMDTFALGLIKAAEIIDDGRIAKFVDDRYASYKTGIGKAIVDGTTSLEELEQYVLTHSEPVMQSGRQEVLETIVNNILFR

SEQ ID NO: 5 - protein, 437 aa, Piromyce sp.
MAKEYFPQIQKIKFEGKDSKNPLAFHYYDAEKEVMGKKMKDWLRFAMAWWHTLCAEGADQFGGGTKSFPWNEGTDAIEIAKQKVDAGFEIMQKLGIPYYCFHDVDLVSEGNSIEEYESNLKAVVAYLKEKQKETGIKLLWSTANVFGHKRYMNGASTNPDFDVVARAIVQIKNAIDAGIELGAENYVFWGGREGYMSLLNTDQKREKEHMATMLTMARDYARSKGFKGTFLIEPKPMEPTKHQYDVDTETAIGFLKAHNLDKDFKVNIEVNHATLAGHTFEHELACAVDAGMLGSIDANRGDYQNGWDTDQFPIDQYELVQAWMEIIRGGGFVTGGTNFDAKTRRNSTDLEDIIIAHVSGMDAMARALENAAKLLQESPYTKMKKERYASFDSGIGKDFEDGKLTLEQVYEYGKKNGEPKQTSGKQELYEAIVAMYQ

SEQ ID NO: 6 - DNA, 2309 bp, Artificial Sequence
  coding sequence of the EsXI Gene with C-terminal 6x-His-Tag and linker fusion
ctcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaagcagttccctcaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacagaaacaaagaaaatgaaaacaaaaaacaacattatatgtactattgcattgaaaggagacatatttatgaaagaattttttcccggcatatcacctgtaaagtttgagggcagagatagtaaaaatccacttagtttcaaatattatgatgccaaaagggtgataatgggcaaaacaatggaggaacatttatcatttgctatggcatggtggcataatctttgtgcctgtggtgtggatatgttcggacagggtactgtcgataaaagttttggtgaaagctccggtactatggagcatgcaagggctaaagtggatgcaggcattgaatttatgaaaaagcttggtataaagtattattgcttccatgatacggatattgtacctgaggatcaggaagatataaatgttaccaatgcacgtttggatgagattacagactatatcttagaaaaaacaaaggataccgatattaaatgtctttggacaacctgcaatatgttcagtaatccaagatttatgaacggtgcaggaagctcaaacagtgcagatgtattttgctttgcagcggcacaggcaaagaaaggtcttgaaaatgccgtaaaacttggagcaaagggatttgtattctggggaggcagagaaggttatgagacacttctaaatacagatatgaagcttgaagaggaaaatatagcaacactctttacaatgtgcagagattatggacgcagtataggctttatgggagatttttatattgagcctaagccgaaggagcctatgaagcatcagtatgattttgatgcggcaactgcaatcggttttttaagaaaatatggacttgataaagatttcaaactaaatattgaggcaaatcacgctacacttgcaggtcatacttttcagcatgagttaagagtatgtgcagtcaacggtatgatggggtcggtagatgccaatcaaggagatacattacttggatgggacactgatcaattccctacaaatgtctatgatactacattggctatgtatgaaatattaaaggcaggcggactccgtggaggtctgaactttgattcaaagaatcgcagaccaagtaatacagccgatgatatgttctatggctttatagcaggtatggacacatttgcacttggacttattaaggcggcggaaattatagaagacggaagaatagatgattttgttaaagaaagatatgcaagttataattcaggaataggtaagaagataagaaacagaaaagtgacactgatagagtgtgccgagtatgccgcaaagcttaaaaagcctgaactgccggaatcaggaagacaggaatatcttgagagcgtagtgaataatatattgttcggaggatctggccatcaccaccatcatcactaatgttcgtcctcgtttagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttg

SEQ ID NO: 7 - DNA, 1347 bp, Artificial Sequence
  S. cerevisiae optimized DNA encoding truncated version of Es-sh_XI with C-terminal fusion of a 6x His TAG
atgaaggaattcttcccaggtatctccccagttaagtttgaaggtagagattctaagaacccattgtccttcaagtactacgatgccaagagagttattatgggtaagaccatggaagaacatttgtcttttgctatggcttggtggcataatttgtgtgcttgtggtgttgatatgttcggtcaaggtactgttgataagtctttcggtgaatcttctggtactatggaacatgctagagctaaagttgatgccggtattgaattcatgaagaagttgggtattaagtactactgcttccacgatactgatatcgttccagaagatcaagaagatatcaacgttaccaatgccagattggacgaaattaccgattacatcttggaaaagactaaggacaccgatatcaagtgtttgtggactacttgtaacatgttctccaacccaagattcatgaacggtgctggttcttctaattctgctgatgttttttgtttcgctgctgctcaagctaaaaagggtttggaaaatgctgttaagttgggtgctaagggttttgttttttggggtggtagagaaggttacgaaactttgttgaacactgacatgaagttggaagaagaaaacattgctaccttgttcaccatgtgtagagattacggtagatccattggtttcatgggtgatttctacattgaacctaagccaaaagaacctatgaagcaccaatacgattttgatgctgctactgctattggtttcttgagaaagtatggtttggacaaggacttcaagttgaacattgaagctaaccatgctactttggctggtcatacttttcaacacgaattgagagtttgtgctgtcaatggtatgatgggttctgttgatgctaatcaaggtgatactttgttgggttgggatactgatcaatttccaactaacgtttacgataccaccttggccatgtacgaaattttgaaagctggtggtttgagaggtggtttaaactttgactctaagaacagaagaccatccaacactgctgatgatatgttttacggtttcattgctggtatggatactttcgctttgggtttgattaaggccgccgaaattattgaagatggtagaattgatgacttcgtcaaagaaagatacgcctcttacaattccggtatcggtaagaagattagaaacagaaaggttaccttgatcgaatgcgctgaatatgctgctaaattgaagaaaccagaattgccagaatccggtagacaagaatatttggaatctgtcgtcaacaacatcttgtttggtggttctggtcatcatcatcaccatcattaa

SEQ ID NO: 8 - protein, 439 aa, Artificial Sequence
  Es-sh_XI N-terminally truncated Eubacterium sabbureum DSM 3986
MKEFFPGISPVKFEGRDSKNPLSFKYYDAKRVIMGKTMEEHLSFAMAWWHNLCACGVDMFGQGTVDKSFGESSGTMEHARAKVDAGIEFMKKLGIKYYCFHDTDIVPEDQEDINVTNARLDEITDYILEKTKDTDIKCLWTTCNMFSNPRFMNGAGSSNSADVFCFAAAQAKKGLENAVKLGAKGFVFWGGREGYETLLNTDMKLEEENIATLFTMCRDYGRSIGFMGDFYIEPKPKEPMKHQYDFDAATAIGFLRKYGLDKDFKLNIEANHATLAGHTFQHELRVCAVNGMMGSVDANQGDTLLGWDTDQFPTNVYDTTLAMYEILKAGGLRGGLNFDSKNRRPSNTADDMFYGFIAGMDTFALGLIKAAEIIEDGRIDDFVKERYASYNSGIGKKIRNRKVTLIECAEYAAKLKKPELPESGRQEYLESVVNNILFG

(19) United States
(12) Patent Application Publication
(10) Pub. No.: US 2014/0377813 A1
(43) Pub. Date: Dec. 25, 2014

(54) PENTOSE FERMENTING MICROORGANISMS

(71) Applicant: CLARIANT PRODUKTE (DEUTSCHLAND) GMBH, Frankfurt am Main (DE)

(72) Inventors: Zdravko Dragovic, Munchen (DE); Christian Gamauf, Munchen (DE); Christoph Reisinger, Munchen (DE); Ulrich Kettling, Munchen (DE)

(21) Appl. No.: 14/376,775

(22) PCT Filed: Feb. 7, 2013
(86) PCT No.: PCT/EP2013/052407

§ 371 Date: Aug. 5, 2014

(30) Foreign Application Priority Data
Feb. 7, 2012 (EP) 12000783.6

(51) Int. Cl. C12N 15/81; C12P 7/06; C12P 7/56; C12P 7/54; C12P 13/04; C12P 35/00; C12P 5/02; C12P 7/16; C12P 7/46; C12P 1/02; C12P 7/20; C12N 9/90; C12P 7/18

(52) U.S. Cl. CPC C12N 15/81; C12N 9/90; C12N 15/815; C12P 7/06; C12P 7/56; C12P 7/54; C12P 13/04; C12P 7/18; C12P 5/026; C12P 7/16; C12P 7/46; C12P 1/02; C12P 7/20; C12P 35/00; C12Y 503/01005
USPC 435/106; 435/233; 536/23.2; 435/254.23; 435/254.2; 435/254.21; 435/254.22; 435/161; 435/139; 435/140; 435/158; 435/167; 435/160; 435/145; 435/171; 435/159; 435/119

(57) ABSTRACT

The invention provides a microbial eukaryotic cell capable of utilizing C5 sugars, in particular xylose. Another objective of the invention is to provide an improved protein sequence to enable eukaryotic cells to degrade C5 sugars. The present invention thus provides protein comprising an amino acid sequence having at least 75% identity, preferably 80% identity, most preferably 90% identity, most highly preferably 95% identity to SEQ ID NO. 2 or SEQ ID NO. 8 and having xylose-isomerase activity in a eukaryotic cell.

### FIELD OF INVENTION

Xylose is a major building block of plant biomass, and finds itself bound in a number of major feedstock in focus by nowadays biorefinery concepts. Examples for such xylose-rich materials include wheat straw, corn stover or wood chips or other wood by-products (Blake A Simmons et al. Genome Biol. 2008, 9(12): 242).

As a consequence a performed hydrolysis of the starting material by enzymatic, chemical or chemo/enzymatic approaches leads to intermediate products rich in xylose, besides other valuable sugars (Deepak Kumar et al. Biotechnol Biofuels. 2011; 4: 27). The efficient utilization of C5 rich sugar solutions in coupled fermentation lines is both crucial and demanding for the applied fermentation strains (Sara Fernandes and Patrick Murray, Bioeng Bugs. 2010; 1(6): 424). Especially C6 yeasts, such as Saccharomyces cerevisiae, that are desired working horses due to the long history of breeding that ended up in traits with extreme ethanol tolerance and high yields for glucose conversion, leave xylose completely untouched, thereby decreasing the potential yield. Several strategies are known to circumvent this limitation. A key step herein appears the successful feeding of the xylose by isomerisation into xylulose and subsequent modification cascade of the non reductive part of the C5 shunt into the regular glycolysis pathway of Saccharomyces cerevisiae. While strength of xylose uptake through membrane by specific transporters and the achievable flux density through the C5 shunt are subject to possible enhancements (David Runquist et al. Microb Cell Fact. 2009: 8: 49), the key isomerization step from xylose to xylulose posts a major problem in the overall process. Two principle pathways are known to perform the step. The first, employing subsequent steps of reduction to xylitol (by xylose reductase) and oxidation (by xylitol dehydrogenase) to xylulose, causes a major imbalance between NADH and NADPH cofactors and leads to increased formation of xylitol under fermentation conditions (Maurizio Bettiga et al. Biotechnol Biofuels. 2008; 1: 16). The alternative direct isomerization by application of xylose isomerase suffers from the lack of availability of xylose isomerase genes combining an active expression in eukaryotic microorganisms (in particular yeasts like Saccharomyces cerevisiae), a high catalytic efficiency, a temperature and a pH optimum adapted to the fermentation temperature and a low inhibition by side products, especially xylitol. One aspect of the present invention is the disclosure of protein sequences and their nucleic acids encoding the same, to fulfill this requirement.

The xylose isomerase pathway is native to bacterial species and to rare yeasts. In contrast to oxidoreductase pathway the isomerase pathway requires no cofactors. The isomerase pathway minimally consists of single enzymes, heterologous xylose isomerases (XI), which directly convert xylose to xylulose. As with the oxidoreductase pathway, the further improvement of the yield can be obtained by coexpression of heterologous xylulose kinase (XK).

First functionally expressed XI was a xylA gene from anaerobic fungus Piromyces sp E2 (Kuyper M. et al. FEMS Yeast Res. 2003; 4(1): 69). The haploid yeast strain with ability to ferment xylose as a sole carbon source under anaerobic conditions was constructed. The majority of xylose isomerases are bacterial proteins and a major obstacle was their expression in yeast. However recent work has demonstrated functional expression in yeast (Table 1). Due to the key importance of the xylose isomerase activity within the concept of C5-fermenting organisms, it is desirable to use optimal xylose isomerases. From the previous reports we learn that Clostridium phytofermentans xylose isomerase provides a low but highest available technical standard with this respect. The improved beneficial properties of xylose isomerases in the scope of this invention are therefor highly desired.

TABLE 1
Examples for xylose isomerases claimed
for the application in yeasts

| Source Organism | Citation |
|---|---|
| Clostridium phytofermentas | WO 2010/000464 |
| Piromyces sp. E2 | EP 1 468 093 B1 |
| Bacteroides thetaiotaomicron | US 2012/0225451 A1 |
| Unknown | WO20110782620 |
| Abiotrophia defectiva | WO/2012/0092720 |

Sugar transport across the membrane does not limit the fermentation of hexose sugars, although it may limit pentose metabolism especially in case of hexose and pentose cofermentations. Several pentose transporter expression studies have been performed.

### BRIEF DESCRIPTION OF THE INVENTION

An objective of the invention is to provide a microbial eukaryotic cell capable of utilizing C5 sugars, in particular xylose. Another objective of the invention is to provide an improved protein sequence to enable eukaryotic cells to degrade C5 sugars.

It was surprisingly found that the protein described by SEQ ID NO. 2 (sequence previously published in NCBI GeneBank accession number ZP_{—}07904696.1) or a N-terminally truncated version devoid of the first 18 amino acids (mktknniictialkgdif) (SEQ ID NO. 8) is functionally expressed in eukaryotic microbial cells, in particular yeasts like Saccharomyces cerevisiae, when these cells are transformed with a vector carrying an expression cassette comprising a DNA sequence coding for said SEQ ID NO. 2 Protein, for example the DNA Molecule described under SEQ ID NO. 1 (previously published in GeneBank as part of Accession Number NZ_AEPW01000073.1 GI:315651683).

The present invention thus provides protein comprising an amino acid sequence having at least 75% identity, preferably 80% identity, most preferably 90% identity, most highly preferably 95% identity to SEQ ID NO. 2 or SEQ ID NO. 8 and having xylose isomerase activity in a eukaryotic cell.

The present invention also provides a DNA molecule comprising a DNA sequence encoding the protein of the invention, wherein the DNA sequence is operably linked to a eukaryotic regulatory sequence.

It was further found, that transformed cells show an increased rate of xylose consumption when compared to the non-transformed cells. The present invention thus also provides a eukaryotic cell expressing the protein of the invention and/or containing the DNA molecule of the invention.

As a further aspect, the fermentation of biomass from xylose carbon source containing media was improved and the amount of metabolites formed by such transformed strains under these conditions was increased compared to transformed controls.

Another aspect of the invention relates to the biocatalytic properties of the expressed protein and its application as biocatalyst in situ or in purified form for the production of isomerized sugar products or intermediates.

### FIGURES

FIG. 1: Xylose utilization pathway.

FIG. 2: Comparison of colony growth between Saccharomyces cerevisiae transformed with Eubacterium saburreum (Es XI), Piromyces sp. (Pi XI) and Clostridum phytofermentas (Cp XI). As negative control the strain transformed with plain expression vector pSCMB454 (Vector) was used. A 1 on the scale corresponds to week growth after 6 days while a 4 corresponds to very strong growth after 6 days.

FIG. 3: Yeast expression plasmid Map.

FIG. 4: Expression Plasmid for EsXI.

FIG. 5: Comparison of culture growth between Saccharomyces cerevisiae transformed with Eubacterium saburreum (Es-sh XI), and Clostridum phytofermentas (Cp XI). As negative control the strain transformed with plain expression vector pSCMB454 (Vector) was used.

FIG. 6: Activity of xylose isomerase in cell extracts of Saccahromyces cerevisiae expressing Eubacterium saburreum (Es-sh XI, diamonds) and Clostridum phytofermentas (Cp XI circless). As negative control the strain transformed with plain expression vector pSCMB454 (Vector) was used. Formulas for linear curve fits (Abs₃₄₀ₙₘ=v*Time+Abs₃ₘᵢₙ) are shown below the legend. For clarity reason only the curve fit was plotted for negative (Vector) control.

FIG. 7: Specific activity of purified xylose isomerases: Eubacterium saburreum Es-sh XI and Clostridum phytofermentas Cp XI. As negative control the reaction mixture without enzyme was used (Buffer only). Specific activity is expressed as % of converted xylose at enzyme to substrate ration (E/S) of 0.05% w/w.

FIG. 8: Determination pH optimum for purified xylose isomerases: Eubacterium saburreum (Es-sh XI, diamonds) and Clostridum phytofermentas (Cp XI, circles). As negative control the reaction mixture without enzyme was used (Buff, triangles). Activity is expressed as % of maximal activity.

FIG. 9: Determination temperature optimum for purified xylose isomerases: Eubacterium saburreum (Es-sh XI, diamonds) and Clostridum phytofermentas (Cp XI, circles). As negative control the reaction mixture without enzyme was used (Buff, triangles). Activity is expressed as % of maximal activity.

FIG. 10: Determination of Km for purified xylose isomerases: Eubacterium saburreum (Es-sh XI) and Clostridum phytofermentas (Cp XI).

### DETAILED DESCRIPTION

Definitions

Xylose isomerase activity is herein defined as the enzymatic activity of an enzyme belonging to the class of xylose isomerases (EC 5.3.1.5), thus catalyzing the isomerisation of various aldose and ketose sugars and other enzymatic side reactions inherent to this class of enzymes. The assignment of a protein to the class of xylose isomerase is either performed based on activity pattern or homology considerations, whatever is more relevant in each case. Xylose isomerase activity can be determined by the use of a coupled enzymatic photometric assay employing sorbitol dehydrogenase.

An expression construct herein is defined as a DNA sequence comprising all required sequence elements for establishing expression of an comprised open reading frame (ORF) in the host cell including sequences for transcription initiation (promoters), termination and regulation, sites for translation initiation, regions for stable replication or integration into the host genome and a selectable genetic marker. The functional setup thereby can be already established or reached by arranging (integration etc.) event in the host cell. In a preferred embodiment the expression construct contains a promoter functionally linked to the open reading frame followed by an optional termination sequence. Regulatory sequences for the expression in eukaryotic cells comprise promoter sequences, transcription regulation factor binding sites, sequences for translation initiation and terminator sequences. Regulatory sequences for the expression in eukaryotic cells are understood as DNA or RNA coded regions staying in functional connection to the transcription and/or translation process of coding DNA strands in eukaryotic cells, when found connected to coding DNA strands alone ore in combination with other regulatory sequences. In the focus of the invention are promoter sequences coupled to the inventive xylose isomerase genes thus enabling their expression in a selected eukaryotic yeast or fungal cell. The combination of eukaryotic promoter and DNA sequences encoding the inventive xylose isomerase is leading to the expression of xylose isomerase in the transformed eukaryotic cell. Preferred promoters are medium to high strength promoters of Saccharomyces cerevisiae, active under fermentative conditions. Examples for such preferred promoters are promoters of the glycolytic pathway or the sugar transport, particularly the promoters of the genes known as PFK1, FBA1, PGK1, ADH1, ADH2, TDH3 as well truncated or mutated variants thereof. Elements for the establishment of mitotic stability are known to the art and comprise S. cerevisiae 2μ plasmid origin of replication, centromeric sequences (CEN), autonomous replicating sequence (ARS) or homologous sequences of any length for the promotion of chromosomal integration via the homologous end joining pathway. Selectable markers include genetic elements referring antibiotic resistance to the host cell. Examples are kan and ble marker genes. Auxotrophy markers complementing defined auxotrophies of the host strain can be used. Examples for such markers to be mentioned are genes and mutations reflecting the leucine (LEU2) or uracil (URA3) pathway, but also xylose isomerase.

Enhanced xylose consumption is herein defined as any xylose consumption rate resulting in cell growth and proliferation, metabolite formation and or caloric energy generation which is increased in comparison to the xylose consumption rate of the non-modified cell (culture) with respect to the considered trait. The consumption rate can be determined for instance phenomenologically by consideration of formed cell density or colony size, by determination of oxygen consumption rate, formation rate of ethanol or by direct measurement of xylose concentration in the growth media over time. Consumption in this context is equivalent to the terms utilization, fermentation or degradation.

Genes involved in the xylose metabolism were described by various authors and encode hexose and pentose transporters, xylulokinase, ribulose-5-phosphate-3-epimerase, ribulose-5-phosphate isomerase, transketolase, transaldolase and homologous genes.

Xylose isomerase expressing cell herein is referred to as a microbial eukaryotic cell which was genetically modified in carrying an expression construct for the expression of the disclosed xylose isomerase. In a preferred embodiment the xylose isomerase expressing cell is a yeast selected from the group of Pichia, Pachysolen, Yarrowia, Saccharomyces, Candida, Arxula, Ashbya, Debaryomyces, Hansenula, Hartaea, Kluyveromyces, Schwanniomyces, Trichosporon, Xanthophylomyces, Schizosaccharomyces, Zygosaccharomyces, most preferably being Saccharomyces cerevisiae.

Detailed Description of the Invention

The present invention provides solutions for the genetic construction of eukaryotic cells with an enhanced xylose metabolism, an improved biomass formation in the presence of xylose and/or improved formation of metabolites. These are desirable properties and present bottlenecks for many industrial production strains, especially production strains of the genus Saccharomyces, to name Saccharomyces cerevisiae as non-limiting example. The invention solves this problem by providing protein and DNA sequences of xylose isomerase genes that are functionally expressed in lower eukaryotic cells, especially yeasts with an outlined example being yeasts of the genus Saccharomyces, again to name Saccharomyces cerevisiae as non-limiting example. The created strain is a xylose isomerase expressing cell showing potentially enhanced xylose consumption. The desired property of xylose isomerase activity produced by the xylose isomerase expressing cell is difficult to realize in a satisfactory manner with means known to the art.

The present invention thus provides a protein comprising an amino acid sequence having at least 75%, such as at least 80% identity, preferably 85% identity, most preferably 90% identity, most highly preferably 95% identity to SEQ ID NO. 2 or SEQ ID NO. 8 and having xylose isomerase activity in a eukaryotic cell. In a preferred embodiment, the protein consists of such an amino acid sequence. In another preferred embodiment, the protein consists of such an amino acid sequence fused to another part of another proteins, preferably parts of such proteins showing high identity levels to known xylose isomerases or demonstrated xylose isomerase activity themselves.

In a preferred embodiment, the protein consists of the sequence of SEQ ID NO. 2, or of an amino acid sequence having at least 75% identity, preferably 80% identity, most preferably 90% identity, most highly preferably 95% identity to SEQ ID NO. 2 and having xylose-isomerase activity in a eukaryotic cell.

Homologous proteins shall also comprise truncated protein sequences with conserved xylose isomerase activity. A dedicated example of such truncated protein sequences is given as SEQ ID NO. 8 or variants thereof showing at least 75%, 80%, 85%, 90% or 95% identityto SEQ ID NO. 8.

The protein of the invention, or a composition containing said protein, is preferably different from a protein or composition that is obtained by expression from a prokaryotic cell. The protein is thus generally one that is obtainable by expression from a eukaryotic cell.

The protein of the invention preferably shows an optimum xylose isomerase activity within a pH range of 7.5 to 8.5, as determined by the method described in the Examples.

Identity levels can be determined by the computer program AlignX, sold in the Vector-NTI-Package by Life™ Technology. The default settings of the package component in version 10.3.0 are applied.

It is clear to the skilled person that high numbers of varying DNA molecules translate to the same protein sequence and shall be covered by the invention as such. The present invention thus also provides a DNA molecule comprising (preferably consisting of) a DNA sequence encoding the protein of the invention, i.e. a protein comprising an amino acid sequence having at least 75%, such as at least 80% identity, preferably 85% identity, most preferably 90% identity, most highly preferably 95% identity to SEQ ID NO. 2 and having xylose isomerase activity in a eukaryotic cell, or a preferred embodiment as illustrated supra, wherein the DNA sequence is operably linked to a eukaryotic regulatory sequence, i.e. a regulatory sequence that allows expression from a eukaryotic cell. Non-limiting examples of DNA sequence are given in SEQ ID NO. 1 or SEQ ID NO. 7. Methods for computational enhancement of a DNA-sequence with respect to protein production levels are known. They nonexclusively include methods employing statistic evaluation of preferred codons (Codon usage tables), mRNA secondary-structure predicting algorithms and knowledge based models based on HMM or NN. In such way optimized DNA sequences calculated from the targeted protein sequence are preferred and included in the invention. Also included in the invention are DNA sequences obtained by recursive or non recursive steps of mutagenesis and selection or screening of improved variants. This is a regular technique for the improvement of DNA and protein sequences and sequences obtained by such methods cannot be excluded from the inventive concept. This shall be seen independent from the question whether the outcoming DNA sequence of such an experiment leaves the translated protein sequence untouched or translates to mutations in them, as long as the levels of identity do not fall below preferably 75%, 80%, 85%, 90% or 95% to SEQ ID NO. 2 or SEQ ID NO. 8, respectively. A preferred embodiment of the invention indeed applies such processes of improvements for the adjustment of the disclosed nucleic acid molecules and protein sequences to the particular problem.

Another aspect of the invention relates to chimeric sequences generated by fusions of parts of the inventive xylose isomerase sequence with parts of other proteins, preferably parts of such proteins showing high identity levels to known xylose isomerases or demonstrated xylose isomerase activity themselves as well as nucleic acid molecules encoding such chimeric proteins. Especially fusions of the N-terminal part of SEQ ID NO. 2 or SEQ ID NO. 8 protein or the 5′-part of the SEQ ID NO. 1 or SEQ ID NO. 7 nucleic acid molecule shall be highlighted as preferred embodiments of the present invention. It has been in the field of vision of the inventors that the step of the xylose isomerization as solved by the invention is one central change required and for the setup of an efficient carbon flux with xylose as starting block further changes the xylose isomerase expressing cell might be necessary. Issues known to the authors include xylose trans-membrane transport, especially uptake from the growth medium, the phosphorylation and the metabolic steps of the C5 shunt (non-oxidative part of the pentose phosphate shunt). Therefore additional changes introduced into the cell, especially those reflecting emendation of the known issues and alter expression levels of genes involved in the xylose metabolism, present a preferred embodiment of the invention. The order of introductions of such changes to the cells, which can be done subsequent or parallel in random or ordered manners, shall not be distinguished at this point and all possible strategies are seen as integral part of and as special embodiments of the invention.

The present invention thus also provides a eukaryotic cell expressing the protein of the invention and/or containing the DNA molecule of the invention. The protein preferably consists of the sequence of SEQ ID NO. 2 or SEQ ID NO. 8. The eukaryotic cell is preferably a yeast cell, more preferably one selected from the group of Pichia, Pachysolen, Yarrowia, Saccharomyces, Candida, Arxula, Ashbya, Debaryomyces, Hansenula, Hartaea, Kluyveromyces, Schwanniomyces, Trichosporon, Xanthophylomyces, Schizosaccharomyces, Zygosaccharomyces, most preferably being Saccharomyces cerevisiae. The invention thus also provides a genetically modified yeast cell comprising an exogenous xylose isomerase gene functional in said yeast cell, preferably wherein the exogenous xylose isomerase gene is operatively linked to promoter and terminator sequences that are functional in said yeast cell. In a preferred embodiment, the exogenous xylose isomerase gene is a DNA molecule according to the invention. The genetically modified yeast cell is preferably selected from the group of Pichia, Pachysolen, Yarrowia, Saccharomyces, Candida, Arxula, Ashbya, Debaryomyces, Hansenula, Hartaea, Kluyveromyces, Schwanniomyces, Trichosporon, Xanthophylomyces, Schizosaccharomyces, Zygosaccharomyces, preferably being Saccharomyces cerevisiae.

The eukaryotic cell having increased levels of xylose isomerase activity is preferably obtained by transformation of a wild type yeast strain with a DNA sequence of the invention. A further aspect of the invention relates to the application of the xylose isomerase of the invention or the xylose isomerase expressing cell of the invention for the production of biochemicals based on xylose containing raw material such as by fermentation of biomass. Biochemicals include biofuels like ethanol or butanol as well as bio-based raw materials for bulk chemicals like lactic acid, itaconic acid to name some examples. A list of possible biochemicals was published by US department of energy. The protein of the invention or the cell of the invention can also be used as a biocatalyst in situ or in purified form for the production of isomerized sugar products or intermediates, preferably for isomerized sugar products.

A further aspect of the invention relates to the use of xylose isomerase enzyme isolated from a eukaryotic, especially a yeast expression host, where said xylose isomerase is free from bacterial contaminants or fragmented of bacterial matter. Possible applications of such xylose isomerase comprise food and feed applications, where presence of mentioned contaminants even at very low level states a risk for product safety. Concerns against a direct application of Eubacterium sabbureum as production host must be raised at this point. The application of the inventive xylose isomerase in a suggested eukaryotic host, preferably a yeast, is clearly advantageous.

### EXAMPLES

1. Identification of Candidate Gene Sequences with Xylose Isomerase Function

For the finding of xylose isomerase sequences within Genebank the program BlastP (Stephen F. Altschul, et al., Nucleic Acids Res. 1997, 25: 3389-3402) at the NCBI genomic BLAST site (http://www.ncbi.nlm.nih.gov/sutils/genom_table.cgi) were chosen. As a test sequence the protein sequence of the Escherichia coli K12 xylose isomerase gene (SEQ ID NO. 3) was taken as query sequence. Standard parameters of the program were not modified and the query was blasted against the bacterial protein databases including Eubacterium saburreum DSM 3986 database built on the results of the shotgun sequence of the organism (accession number NZ_AEPW01000000). Sequences with significant homology level over the whole sequence length were taken into account. The search revealed a number of potential candidate genes which were subsequently cloned into S. cerevisiae and tested for functional expression. All such candidate genes were treated as entry ZP_{—}07904696.1 (SEQ ID NO. 2) which is xylose isomerase (EsXI) from Eubacterium saburreum DSM 3986 as described in the following paragraphs. The linked coding sequence entry (NZ_AEPW01000073 REGION: 2583 . . . 3956: SEQ ID NO. 1) was taken as basis for the construction of cloning primers.

2. Amplification of Eubacterium saburreum DSM 3986 Xylose Isomerase Gene (EsXI)

Methods for manipulation of nucleic acid molecules are generally known to the skilled person in the field and are here introduced by reference (1. Molecular cloning: a laboratory manual, Joseph Sambrook, David William Russell; 2. Current Protocols in Molecular Biology, Last Update: Jan. 11, 2012. Page Count: approx. 5300. Print ISSN: 1934-3639). Genomic template DNA of Eubacterium saburreum DSM 3986 was purchased from DSMZ (Deutsche Stammsammlung für Mikroorganismen und Zellkulturen). Flanking Primer pairs were designed to match the N- and C-terminal ending of SEQ ID NO. 1. For the amplification of an N-terminally truncated version of SEQ ID NO. 1 the binding region of the sense-primer was shifted 54 bp downstream (starting with A55). The PCR reaction is set up using Finnzymes Phusion™ High Fidelity Polymerase (HF-Buffer system) following the recommendations of the supplier for dNTP, primer and buffer concentrations. The amplification of the PCR products is done in an Eppendorf Thermocycler using the standard program for Phusion Polymerase (98° C. 30″ initial denaturation followed by 35 cycles of 98° C. (20″)-60° C. (20″)-72° C. (1′20″) steps and a final elongation phase at 72° C. for 10 minutes. The PCR products of expected size are purified by preparative ethidium bromide stained TAE-Agarose gel electrophoresis and recovered from the Gel using the Promga Wizard SV-PCR and Gel Purification Kit. For the fusion of a C-terminal 6×His-Tag the primary PCR-products are used as template for re-amplification of the whole DNA fragment using an extended reverse Primer with corresponding 5′ extension, under identical conditions (6×HIS-Tag fusion PCR). The PCR products obtained are again purified by Agarose Gel Electrophoresis and recovered using the Promga Wizard SV-PCR and Gel Purification Kit. It contains the C-terminal 6×-His TAG Version of the EsXI-gene or a C-terminal 6×-His TAG Version of the (truncated EsXI) Es-sh XI-gene-gene, respectively.

Amplification of codon-optimized xylose isomerase genes was done from optimized gene-templates ordered from Geneart Regensburg, Germany. Optimization algorithms for sequence optimization were used as provided by the company.

3. Cloning of the EsXI and Es-shXI ORF into Saccharomyces cerevisiae Expression Plasmid

A plasmid preparation of the pSCMB454 plasmid isolated from an Escherichia coli culture was linearized by restriction with XmnI endonuclease and digested fragments separated from unprocessed species by agarose gel electrophoresis. The linearized vector-backbone was recovered from the gel following the instructions of the Promga Wizard SV-PCR and Gel Purification Kit. The amplified PCR product is cloned into the XmnI digested vector-backbone using standard cloning methods. Transformation was performed into chemically competent Escherichia coli W Mach1 cells according to the supplier's protocol. Transformants were grown over night on LB-Ampicillin plates and tested for correctness by plasmid MINI-prep and control digestion as well as DNA sequencing. A larger quantity of plasmid DNA was prepared from a confirmed clone using the Promega PureYield™ Plasmid Midiprep System. An example of the sequence of the resulting expression cassette including GPD-promoter-sequence and cyc1 terminator is given in SEQ ID NO. 6.

4. Transformation in Saccharomyces cerevisiae

Saccharomyces cerevisiae strain ATCC 204667 (MATa, ura3-52, mal GAL+, CUP(r)) was used as host for all transformation experiments.

Transformation is performed using standard methods known to those skilled in the art (e.g. see Gietz, R. D. and R. A. Woods. (2002) Transformation of yeast by the LiAc/ss carrier DNA/PEG method. Methods in Enzymology 350: 87-96). An intact version of the S. cerevisiae ura3 gene contained in the expression vector was used as selection marker and transformants are selected for growth on minimal medium without uracil. Minimal medium consisted of 20 g·l⁻¹ glucose, 6.7 g·l⁻¹ yeast nitrogen base without amino acids, 40 mg·l⁻¹ L-tyrosine, 70 mg·l⁻¹ L-phenylalanine, 70 mg·l⁻¹ L-tryptophane, 200 mg·l⁻¹ L-valine and 50 mg·l⁻¹ each of adenin hemisulfate, L-arginine hydrochloride, L-histidine hydrochloride monohydrate, L-isoleucine, L-leucine, L-lysine hydrochloride, L-methionine, L-serine and L-threonine. The pH was adjusted to 5.6 and 15 g·l⁻¹ agar is added for solid media.

5. Growth of Xylose Isomerases Expressing Saccharomyces Strains on Xylose Media

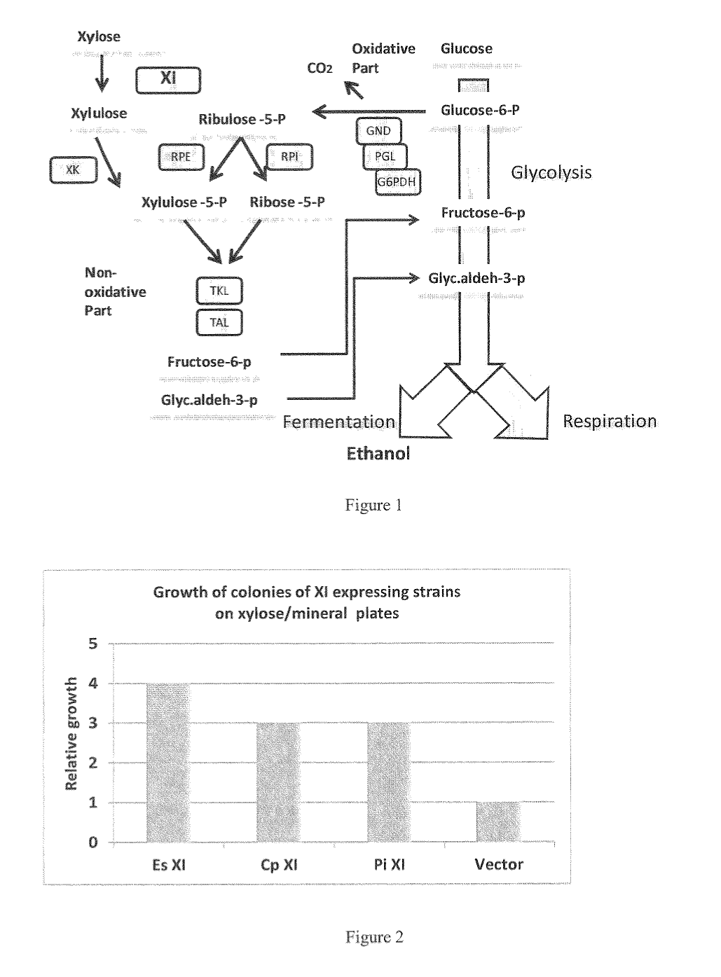

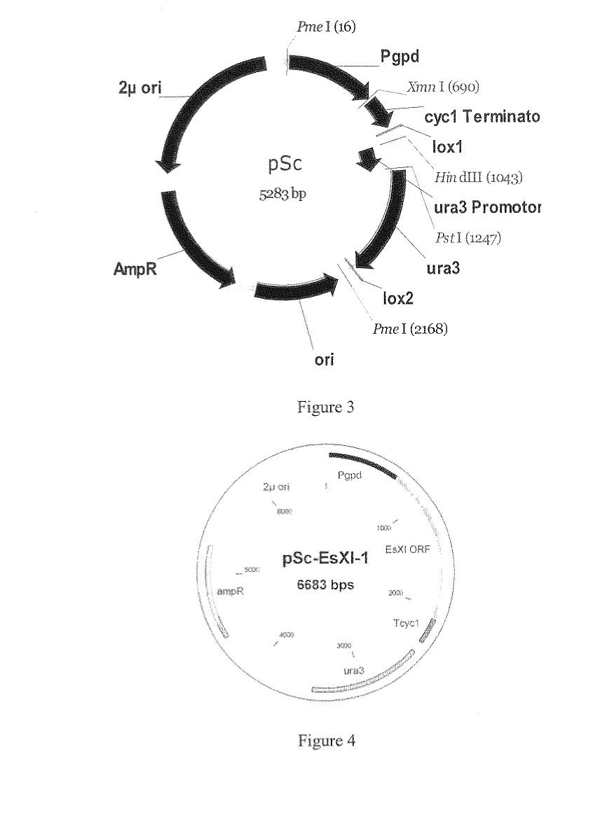

A) Single colons of Saccharomyces strains transformed with expression vector for xylose isomerase from Eubacterium saburreum (Es XI), and Clostridum phytofermentas (Cp XI) as well as plain expression vector pSCMB454 were transferred on minimal medium plates with glucose as single carbon source. Single colonies were transferred then on minimal media plates with xylose as single carbon source (20 g·l⁻¹) and incubated at 30° C. After 7 days only the transformants with xylose isomerase expression vectors were visibly growing (FIG. 2).

Examining the average colony size of Saccharomyces strains expressing different xylose isomerases indicates that strongest effect was observed with Es XI. Cp XI and Pi XI had similar strong effect in this physiological test but the effect was noticeably weaker than for Es XI. Negative control, Saccharomyces strain transformed pSCMB454 only (plain vector), showed only week background growth indistinguishable from background growth of non-transformed Saccharomyces.

B) The growth of the strains was also assessed on liquid medium. Minimal medium with 20 g·l⁻¹ xylose as single carbon source, adjusted to pH 5.6 was inoculated with single colony. After 7 days the cultures were aliquoted, stored at −80° C. and used as starter cultures for growth experiment. The growth experiment was performed on the same minimal medium and was inoculated with the starter cultures. Incubation was done for 10 days in shaken flasks at 250 rpm, 30° C. Growth was assessed by measuring OD₆₀₀ₙₘ (FIG. 5).

As can be deduced from FIG. 5, growth of the Saccharomyces strain transformed with Es XI is slightly stronger than with strain with Cp XI. Error bars present one standard deviation of 3 measured shaken flasks per strain and indicate statistical significance of the measurement.

6. Preparation of Yeast Cell Free Extracts

Single colonies of the Saccharomyces strains expressing Es XI, Cp XI, were transferred to minimal medium containing 20 g·l⁻¹ xylose, 6.7 g·l⁻¹ yeast nitrogen base without amino acids. pH was adjusted to 5.6. The cultures were incubated aerobically at 30° C., 250 rpm for 7 to 10 days. Cells were harvested by centrifugation and washed once with sterile water at RT, resuspended in sterile dd water with OD₆₀₀ₙₘ>200 and frozen at −80° C.

Frozen cell suspension was thawed on ice and adjusted to OD₆₀₀ₙₘ=200, 100 μl of NMDT Buffer stock (250 mM NaCl, 10 mM MnCl₂, 1 mM DTT, 250 mM Tris/HCl pH 7.5) and 11 μl PMSF stock (100 mM in isopropanol) were added on 1000 μl of cell suspension. 500 μl of buffered cell suspension was transferred to Precellys-Glass Kit 0.5 mM (Order #91 PCS VK05) and mechanically lysed in Precellys 24 (Peqlab) homogenisator. The lysis was done 2×15 sec at 5500 rpm. The cell debris was removed by centrifugation at 13,200 g/4° C. Obtained lysate was aliquoted, frozen at liquid nitrogen and stored at −80° C.

7. Assays for Measurement for Xylose Isomerase Activity

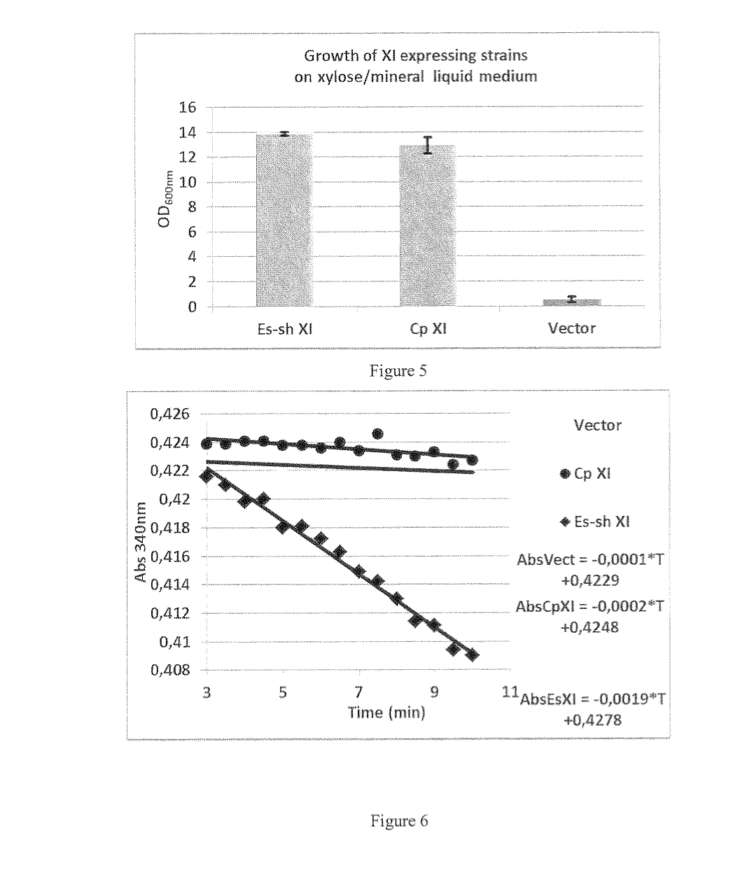

A) For some measurements of xylose isomerase activity we have applied sorbitol dehydrogenase (SD) based spectrophotometrical assay. As product of xylose isomerase, isomeric sugar xylulose is formed. In the enzymatic assay, amount of produced xylulose was measured. For measurement of isomerase activity in total cell lysates, enzymatic assay was performed in form of coupled XI-SD assay (FIG. 6). For all other experiments the enzymatic assay was performed in two steps, xylose isomerization as first step followed by xylulose concentration determination as second step. In two step protocol inactivation of xylose isomerase was performed (95° C., 10 min) after the first step. Composition of enzymatic assay mixture is given below:

| Components | Final concentration | |
|---|---|---|
| Tris-Cl pH 7.5 | 100 | mM |
| MgCl₂ | 105 | mM |
| MnCl₂ | 10 | mM |
| DTT | 1 | mM |
| NADH | 0.25 | mM |
| Sorbitol Dehydrogenase | 2 | U/ml |
| (Enzymstock 100 U/ml) Sigma #S3764 | | |
| Xylose | 1% | (w/v) |

| | |
|---|---|
| ddH₂O | / |

Assay was performed in 96 well microtiter plates and kinetic was followed at 340 nm.

Assessing enzyme activity in cell extracts of Saccharomyces cerevisiae expressing different XIs (FIG. 6) showed that the highest activity was obtained with Eubacterium saburreum XI. Clostridum phytofermentas XI activity was measurable and above background level of the XI-free cell extract but significantly lower if compared to other two extracts.

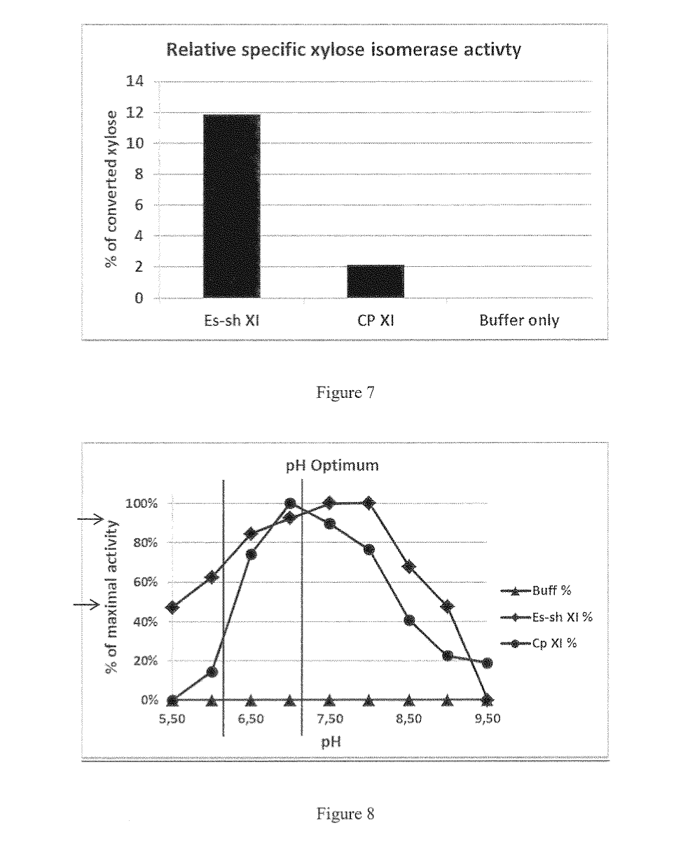

B) For some measurements of xylose isomerase activity an HPLC based method was applied. The amount of produced xylulose was measured indirectly over xylose concentration decrease (FIG. 7). The measurement was performed with H column and Dionex Ultimate 3000 instrument.

8. Expression of Xylose Isomerase in E. coli, Xylose Isomerase Purification and Activity Measurements

All xylose isomerases were expressed in E. coli K12 Top10 cells under arabinose inducible promoter by using standard molecular biology technics. XI expression was induced with 0.02% arabinose at 25° C. and 200 rpm for 14 h. Cultures were harvested by centrifugation, supernatants discarded and cells were resuspended in 100 mM phosphate buffer pH 7.0 at OD₆₀₀ₙₘ between 200 and 300. The cells were lysed by ultrasonification according standard purification methods. Lysed cells were centrifuged for 30 min at 20,000 g at 4° C. Cleared supernatants were aliquoted and frozen at −80° C. Prior purification the lysates were thawed on ice and imidazole was added to 10 mM final. Purification was done on 500 μl Ni-NTA spin columns (Biorad). The columns were equilibrated with 100 mM phosphate butter pH 7.0, and cell lysates were loaded on columns. The columns were washed once with 100 mM phosphate pH 7.0 with 20 mM imidazole and eluted with the same buffer containing 250 mM imidazole. Imidazole removal and buffer exchange (from phosphate to Tris-Cl was done with Micro Bio-Spin columns (Biorad) according instruction manual. SDS-PAGE analysis was done on 10% gels (Birad Criterion XT) according instruction manual. All proteins were purified to homogeneity (>99%). Protein concentration was determined by Bradford reagent from Biorad according instruction manual. Bovine serum albumin was used as a standard. All purified proteins were obtained at final concentration at approx. 2 g/l.

Initial activity measurements of purified proteins was done with end-point HPLC based method (please see above). The measurements were performed at enzyme to substrate ration (E/S ratio) from 0.05%, 60° C. and 2 h. After isomerization the reactions were inactivated as previously described.

The assay was used to get insight in specific activity of purified enzymes. As shown in FIG. 7 the highest specific activity was observed with Eubacterium saburreum XI (11.9%). The lowest specific activity was obtained with Clostridum phytofermentas XI (2.1%). The obtained data are consistent with activity measurements of XI in crude cell extracts. In both cases two novel isomerases described in this invention had higher activity than referent Cp XI.

9. Determination of pH Optimum for Purified Xylose Isomerases

pH optimum for purified XIs was determined with previously described end point sorbitol dehydrogenase based enzyme assay. The described two step protocol was used. As measure for isomerase activity the amount of oxidized NADH (NAD⁺; followed as decrease at 340 nm) at reaction endpoint was used. The amount of oxidized NADH is equimolar to amount of xylulose molecules formed during isomerization step. The care was taken that NADH was not depleted in any of reactions used for pH opt determination. Two buffer systems were used for pH optimum determination: BisTris for pH 5.5-7.5 and Tris from 7.5-9.5. Comparison of enzyme activities in the two buffer systems was done at pH 7.5. No significant differences were observed.

Determined pH optimum, as shown in FIG. 8, demonstrates several differences between referent Cp XI and two novel XIs described in this invention. First: pH optimum for Cp XI is neutral (pH=7.0) and pH optimum for Es XI and Cp XI is in alkalic region (pH=8.0). Second: residual activity of Es XI (pH=5.5) is at 50% (lower arrow) of maximal activity. Residual activity of Cp XI at pH=5.5 virtually equals zero. Third: two novel XIs form relatively broad peak between pH 7.0 (>90%, upper arrow) and pH 8.0 (=100%). In comparison Cp XI is retains <80% of activity at pH=8.0.

10. Determination of Temperature Optimum for Purified Xylose Isomerases

Temperature optimum for purified XIs was determined with previously described end point enzyme assay. Also in this experiment the care was taken that NADH was not depleted in any of reactions used for T. opt. determination. Temperature gradients were generated with common laboratory PCR cyclers (Eppendorf).

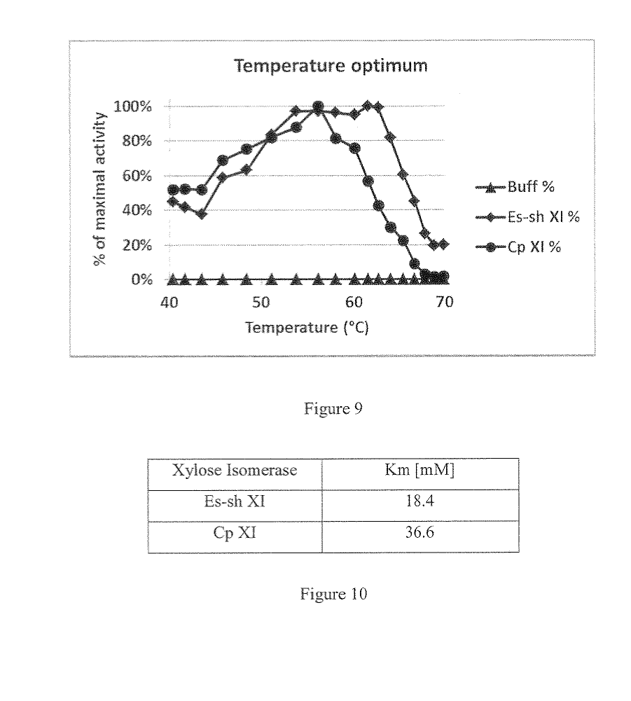

Determination of temperature optimum (FIG. 9) revealed several differences between referent Cp XI and in this invention described Es XI I. First: temperature optimum for Cp Xi is defined with relatively sharp peak at 56.2° C. Es XI shows significantly broader peaks ranging from 53.8° C. to 61.6° C. Second: Activity of Es XI at 67° C. is around 50% and for Cp X virtually equals zero. Taken together the T.opt. data show that the inventive XIs described in this invention posses significantly higher temperature stability than reference Cp XI.

11. Determination of Km for Purified Xylose Isomerases

Km values were determined with the enzyme assay described in previous examples. For the experiment xylose isomerases purified from E. coli were used.

Determination of Km for the purified Xylose isomerases revealed Km for Es-sh XI of 18.4 mM. Km for Cp XI (Km=36.6 mM). (FIG. 10)

Sequence listing

SEQ ID NO 1: Sequence of Eubacterium sabbureum DSM 3986 DNA sequence encoding xylose
isomerase (NZ_AEPW01000073.1 GI:315651683)
gtgaaaacaaaaaacaacattatatgtactattgcattgaaaggagacatatttatgaaagaattttttcccggcatatcacctgtaaa
gtttgagggcagagatagtaaaaatccacttagtttcaaatattatgatgccaaaagggtgataatgggcaaaacaatggaggaacatt
tatcatttgctatggcatggtggcataatctttgtgcctgtggtgtggatatgttcggacagggtactgtcgataaaagttttggtgaa
agctccggtactatggagcatgcaagggctaaagtggatgcaggcattgaatttatgaaaaagcttggtataaagtattattgcttcca
tgatacggatattgtacctgaggatcaggaagatataaatgttaccaatgcacgtttggatgagattacagactatatcttagaaaaaa
caaaggataccgatattaaatgtctttggacaacctgcaatatgttcagtaatccaagatttatgaacggtgcaggaagctcaaacagt
gcagatgtattttgctttgcagcggcacaggcaaagaaaggtcttgaaaatgccgtaaaacttggagcaaagggatttgtattctgggg
aggcagagaaggttatgagacacttctaaatacagatatgaagcttgaagaggaaaatatagcaacactctttacaatgtgcagagatt
atggacgcagtataggctttatgggagatttttatattgagcctaagccgaaggagcctatgaagcatcagtatgattttgatgcggca
actgcaatcggttttttaagaaaatatggacttgataaagatttcaaactaaatattgaggcaaatcacgctacacttgcaggtcatac
ttttcagcatgagttaagagtatgtgcagtcaacggtatgatggggtcggtagatgccaatcaaggagatacattacttggatgggaca
ctgatcaattccctacaaatgtctatgatactacattggctatgtatgaaatattaaaggcaggcggactccgtggaggtctgaacttt
gattcaaagaatcgcagaccaagtaatacagccgatgatatgttctatggctttatagcaggtatggacacatttgcacttggacttat
taaggcggcggaaattatagaagacggaagaatagatgattttgttaaagaaagatatgcaagttataattcaggaataggtaagaaga
taagaaacagaaaagtgacactgatagagtgtgccgagtatgccgcaaagcttaaaaagcctgaactgccggaatcaggaagacaggaa
tatcttgagagcgtagtgaataatatattgttcggataa
SEQ ID NO. 2: Protein Sequence of translated Eubacterium sabbureum DSM 3986 DNA
sequence encoding xylose isomerase (ZP_07904696.1) (EsXI)
mktknniictialkgdifmkeffpgispvkfegrdsknplsfkyydakrvimgktmeehlsfamawwhnlcacgvdmfgqgt
vdksfgessgtmeharakvdagiefmkklgikyycfhdtdivpedqedinvtnarldeitdyilektkdtdikclwttcnmf
snprfmngagssnsadvfcfaaaqakkglenavklgakgfvfwggregyetllntdmkleeeniatlftmcrdygrsigfmg
dfyiepkpkepmkhqydfdaataigflrkygldkdfklnieanhatlaghtfqhelrvcavngmmgsvdanqgdtllgwdtd
qfptnvydttlamyeilkagglrgglnfdsknrrpsntaddmfygfiagmdtfalglikaaeiiedgriddfvkeryasyns
gigkkirnrkvtliecaeyaaklkkpelpesgrqeylevvnnilfg*
SEQ ID NO 3: Esccheria coli xylose isomerase (Protein)
mqayfdqldryryegskssnplafrhynpdelvlgkrmeehlrfaacywhtfcwngadmfgvgafnrpwqqpgealalakrk
advafeffhklhvpfycfhdvdvspegaslkeyinnfaqmvdvlagkqeesgvkllwgtancftnprygagaatnpdpevfs
waatqvvtameathklggenyvlwggregyctllntdlrqereqlgrfmqmvvehkhkigfqgtlliepkpqeptkhqydyd
aatvygflkqfglekeiklnieanhatlaghsfhheiataialglfgsvdanrgdaqlgwdtdqfpnsveenalvmyeilka
ggfttgglnfdakvrrqstdkydlfyghigamdtmalalkiaarmiedgeldkriaqrysgwnselgqqilkgqmsladlak
yaqehhlspvhqsgrqeqlenlynhylfdk*
SEQ ID NO. 4: Clostridium phytofermentas xylose isomerase (Protein) (CpXI)
mknyfpnvpevkyegpnstnpfafkyydankvvagktmkehcrfalswwhtlcaggadpfgvttmdrtygnitdpmelaka
kvdagfelmtklgieffcfhdadiapegdtfeeskknlfeivdyikekmdqtgikllwgtannfshprfmhgastscnadv
fayaaakiknaldatiklggkgyvfwggregyetllntdlgleldnmarlmkmaveygrangfdgdfyiepkpkeptkhqy
dfdtavlaflrkyglekdfkmnieanhatlaghtfehelamarvngafgsvdanqgdpnlgwdtdqfptdvhsatlamlev
lkaggftngglnfdakvrrgsfefddiaygyiagmdtfalglikaaeiddgriakfvddryasyktgigkaivdgttslee
leqvylthsepvmqsgrpevletivnnilfr*
SEQ ID NO: 5: Piromyce sp. xylose isomerase (Protein-PI_XI)
makeyfpqiqkiklegkdsknplafhyydaekevmgkkmkdwlrfamawwhtlcaegadqfgggtksfpwnegtdaieia
kqkvdagfeimqklgipyycfhdvdlvsegnsieeyesnlkavvaylkekqketgikllwstanvfghkrymngastnpd
fdvvaraivqiknaidagielgaenyvfwggregymsllntdqkrekehmatmltmardyarskgfkgtfliepkpmept
khqydvdtetaigflkahnldkdfkvnievnhatlaghtfehelacavdagmlgsidanrgdyqngwdtdqfpidqyelv
qawmeiirgggfvtggtnfdaktrrnstdlediiiahvsgmdamaralenaakllqespytkmkkeryasfdsgigkdfe
dgkltleqvyeygkkngepkqtsgkqelyeaivamyq*
SEQ ID NO. 6: EsXI Expression cassette (BOLD CAPITALS: coding sequence
of the EsXI Gene with C-terminal 6x-His-Tag and linker fusion;
SMALL CAPITALS : GPD-promoter; underlinded: remains of XnmI site;
italic: CYC1 terminator)
CTCGCCATTTCAAAGAATACGTAAATAATTAATAGTAGTGATTTTCCTAACTTTATTTAGTCAAAAAAT
TAGCCTTTTAATTCTGCTGTAACCCGTACATGCCCAAAATAGGGGGCGGGTTACACAGAATATATAACA
TCGTAGGTGTCTGGGTGAACAGTTTATTCCTGGCATCCACTAAATATAATGGAGCCCGCTTTTTAAGCT
GGCATCCAGAAAAAAAAAGAATCCCAGCACCAAAATATTGTTTTCTTCACCAACCATCAGTTCATAGGT
CCATTCTCTTAGCGCAACTACAGAGAACAGGGGCACAAACAGGCAAAAAACGGGCACAACCTCAATGGA
GTGATGCAACCTGCCTGGAGTAAATGATGACACAAGGCAATTGACCCACGCATGTATCTATCTCATTTT
CTTACACCTTCTATTACCTTCTGCTCTCTCTGATTTGGAAAAAGCTGAAAAAAAAGGTTGAAAGCAGTT
CCCTCAAATTATTCCCCTACTTGACTAATAAGTATATAAAGACGGTAGGTATTGATTGTAATTCTGTAA
ATCTATTTCTTAAACTTCTTAAATTCTACTTTTATAGTTAGTCTTTTTTTTAGTTTTAAAACACCAAGA
ACTTAGTTTCGAATaaacacacagaaacaaagaaaATGAAAACAAAAAACAACATTATATGTACTATTG
CATTGAAAGGAGACATATTTATGAAAGAATTTTTTCCCGGCATATCACCTGTAAAGTTTGAGGGCAGAG
ATAGTAAAAATCCACTTAGTTTCAAATATTATGATGCCAAAAGGGTGATAATGGGCAAAACAATGGAGG
AACATTTATCATTTGCTATGGCATGGTGGCATAATCTTTGTGCCTGTGGTGTGGATATGTTCGGACAGG
GTACTGTCGATAAAAGTTTTGGTGAAAGCTCCGGTACTATGGAGCATGCAAGGGCTAAAGTGGATGCAG
GCATTGAATTTATGAAAAAGCTTGGTATAAAGTATTATTGCTTCCATGATACGGATATTGTACCTGAGG
ATCAGGAAGATATAAATGTTACCAATGCACGTTTGGATGAGATTACAGACTATATCTTAGAAAAAACAA
AGGATACCGATATTAAATGTCTTTGGACAACCTGCAATATGTTCAGTAATCCAAGATTTATGAACGGTG
CAGGAAGCTCAAACAGTGCAGATGTATTTTGCTTTGCAGCGGCACAGGCAAAGAAAGGTCTTGAAAATG
CCGTAAAACTTGGAGCAAAGGGATTTGTATTCTGGGGAGGCAGAGAAGGTTATGAGACACTTCTAAATA
CAGATATGAAGCTTGAAGAGGAAAATATAGCAACACTCTTTACAATGTGCAGAGATTATGGACGCAGTA
TAGGCTTTATGGGAGATTTTTATATTGAGCCTAAGCCGAAGGAGCCTATGAAGCATCAGTATGATTTTG
ATGCGGCAACTGCAATCGGTTTTTTAAGAAAATATGGACTTGATAAAGATTTCAAACTAAATATTGAGG
CAAATCACGCTACACTTGCAGGTCATACTTTTCAGCATGAGTTAAGAGTATGTGCAGTCAACGGTATGA
TGGGGTCGGTAGATGCCAATCAAGGAGATACATTACTTGGATGGGACACTGATCAATTCCCTACAAATG
TCTATGATACTACATTGGCTATGTATGAAATATTAAAGGCAGGCGGACTCCGTGGAGGTCTGAACTTTG
ATTCAAAGAATCGCAGACCAAGTAATACAGCCGATGATATGTTCTATGGCTTTATAGCAGGTATGGACA
CATTTGCACTTGGACTTATTAAGGCGGCGGAAATTATAGAAGACGGAAGAATAGATGATTTTGTTAAAG
AAAGATATGCAAGTTATAATTCAGGAATAGGTAAGAAGATAAGAAACAGAAAAGTGACACTGATAGAGT
GTGCCGAGTATGCCGCAAAGCTTAAAAAGCCTGAACTGCCGGAATCAGGGTGTGCCGAGTATGCCGCAA
AGCTTAAAAAGCCTGAACTGCCGGAATCAGGAAGACAGGAATATCTTGAGAGCGTAGTGAATAATATAT
TGTTCGGAGGATCTGGCCATCACCACCATCATCACTAAtgttcgtcctcgtttagttatgtcacgctta
cattcacgccctccccccacatccgctctaaccgaaaaggaaggagttagacaacctgaagtctaggtc
cctatttatttttttatagttatgttagtattaagaacgttatttatatttcaaatttttctttttttt
ctgtacagacgcgtgtacgcatgtaacattatactgaaaaccttgcttgagaaggttttgggacgctcg
aaggctttaatttg
SEQ ID NO. 7: S. cerevisiae optimized DNA encoding truncated version of
Es-sh_XI with C-terminal fusion of a 6x His TAG.
atgaaggaattcttcccaggtatctccccagttaagtttgaaggtagagattctaagaacccattgtccttcaagtactacgat
gccaagagagttattatgggtaagaccatggaagaacatttgtcttttgctatggcttggtggcataatttgtgtgcttgtggt
gttgatatgttcggtcaaggtactgttgataagtctttcggtgaatcttctggtactatggaacatgctagagctaaagttgat
gccggtattgaattcatgaagaagttgggtattaagtactactgcttccacgatactgatatcgttccagaagatcaagaagat
atcaacgttaccaatgccagattggacgaaattaccgattacatcttggaaaagactaaggacaccgatatcaagtgtttgtgg
actacttgtaacatgttctccaacccaagattcatgaacggtgctggttcttctaattctgctgatgttttttgtttcgctgct
gctcaagctaaaaagggtttggaaaatgctgttaagttgggtgctaagggttttgttttttggggtggtagagaaggttacgaa
actttgttgaacactgacatgaagttggaagaagaaaacattgctaccttgttcaccatgtgtagagattacggtagatccatt
ggtttcatgggtgatttctacattgaacctaagccaaaagaacctatgaagcaccaatacgattttgatgctgctactgctatt
ggtttcttgagttaagtatggtttggacaaggacttcaagttgaacattgaagctaaccatgctactttggctggtcatacttt
tcaacacgaattgagagtttgtgctgtcaatggtatgatgggttctgttgatgctaatcaaggtgatactttgttgggttggga
tactgatcaatttccaactaacgtttacgataccaccttggccatgtacgaaattttgaaagctggtggtttgagaggtggttt
aaactttgactctaagaacagaagaccatccaacactgctgatgatatgttttacggtttcattgctggtatggatactttcgc
tttgggtttgattaaggccgccgaaattattgaagatggtagaattgatgacttcgtcaaagaaagatacgcctcttacaattc
cggtatcggtaagaagattagaaacagaaaggttaccttgatcgaatgcgctgaatatgctgctaaattgaagaaaccagaatt
gccagaatccggtagacaagaatatttggaatctgtcgtcaacaacatcttgtttggtggttctggtcatcatcatcaccatca
ttaa
SEQ ID NO: 8: Es-sh_XI N-terminally trucated Eubacterium sabbureum DSM 3986
mkeffpgispvkfegrdsknplsfkyydakrvimgktmeehlsfamawwhnlcacgvdmfgqgtvdksfgessgtmehara
kvdagiefmkklgikyycfhdtdivpedqedinvtnarldeitdyilektkdtdikclwttcnmfsnprfmngagssnsad
vfcfaaaqakkglenavklgakgfvfwggregyetllntdmkleeeniatlftmcrdygrsigfmgdfyiepkpkepmkhq
ydfdaataigflrkygldkdfklnieanhatlaghtfqhelrvcavngmmgsvdanqgdtllgwdtdqfptnvydttlamy
eilkagglrgglnfdsknrrpsntaddmfygfiagmdtfalglikaaeiiedgriddfvkeryasynsgigkkirnrkvtl
iecaeyaaklkkpelpesgrqeylesvvnnilfg*

What is claimed is:

1. A protein comprising an amino acid sequence having at least 75% identity to SEQ ID NO. 2 and having xylose-isomerase activity in a eukaryotic cell.

2. The protein of claim 1, wherein the protein consists of the sequence of SEQ ID NO. 2, or of an amino acid sequence having at least 75% identity to SEQ ID NO. 2 and having xylose-isomerase activity in a eukaryotic cell.

3. The protein of claim 1, wherein the protein consists of the sequence of SEQ ID NO. 8, or of an amino acid sequence having at least 75% identity to SEQ ID NO. 8 and having xylose-isomerase activity in a eukaryotic cell.

4. The protein of claim 1, showing optimum xylose-isomerase activity within a pH range of 7.5 to 8.5.

5. The protein of claim 1, obtainable by expression from a eukaryotic cell.

6. A DNA molecule comprising a DNA sequence encoding a protein as defined in claim 1, wherein the DNA sequence is operably linked to a eukaryotic regulatory sequence.

7. The DNA molecule of claim 6, wherein the DNA molecule consists of the sequence of SEQ ID NO. 1 or SEQ ID NO. 7.

8. A eukaryotic cell expressing a protein of claim 1 and/or containing the DNA molecule according to claim 6.

9. The eukaryotic cell of claim 8, wherein the eukaryotic cell is a yeast cell, selected from the group consisting of Pichia, Pachysolen, Yarrowia, Saccharomyces, Candida, Arxula, Ashbya, Debaryomyces, Hansenula, Hartaea, Kluyveromyces, Schwanniomyces, Trichosporon, Xanthophylomyces, Schizosaccharomyces, and Zygosaccharomyces.

10. A genetically modified yeast cell comprising an exogenous xylose isomerase gene functional in said yeast cell, wherein the exogenous xylose isomerase gene is operatively linked to promoter and terminator sequences that are functional in said yeast cell, leading to the expression of a protein according to claim 1.

11. The genetically modified yeast cell of claim 10 wherein the exogenous xylose isomerase gene is a DNA molecule of claim 6.

12. The genetically modified yeast cell of claim 10, wherein the genetically modified yeast cell is selected from the group consisting of Pichia, Pachysolen, Yarrowia, Saccharomyces, Candida, Arxula, Ashbya, Debaryomyces, Hansenula, Hartaea, Kluyveromyces, Schwanniomyces, Trichosporon, Xanthophylomyces, Schizosaccharomyces, and Zygosaccharomyces.

13. A eukaryotic cell having increased levels of xylose isomerase activity obtained by transformation of a wild type yeast strain with a DNA sequence according to claim 6.

14. The eukaryotic cell of claim 8, wherein the expressed protein consists of the sequence of SEQ ID NO. 2 or SEQ ID NO. 8.

15. The eukaryotic cell of claim 13, wherein the yeast strain is selected from the group consisting of Pichia, Pachysolen, Yarrowia, Saccharomyces, Candida, Arxula, Ashbya, Debaryomyces, Hansenula, Hartaea, Kluyveromyces, Schwanniomyces, Trichosporon, Xanthophylomyces, Schizosaccharomyces, and Zygosaccharomyces.

16. The protein of claim 1, wherein said protein is used for fermentation of biomass from a xylose-carbon source containing media.

17. The protein of claim 1, wherein said protein is used as a biocatalyst in situ or in purified form for the production of isomerized sugar products or intermediates, preferably for isomerized sugar products.

18. The DNA molecule of claim 6, wherein said DNA molecule is used for transformation of a eukaryotic cell.

19. The DNA molecule of claim 6, wherein said DNA molecule is used for transformation of a eukaryotic cell and wherein said transformation results in a eukaryotic cell of claim 8.

20. The eukaryotic cells of claim 8 wherein said cells are used for achieving an increased rate of xylose consumption.

21. A process for producing ethanol from xylose or a glucose-xylose mixture using a yeast, wherein said yeast expresses the protein of claim 1.

22. A process for producing a fermentation product selected from the group consisting of lactic acid, acetic acid, succinic acid, amino acids, 1,3-propane-diol, ethylene, glycerol, β-lactam antibiotics, cephalosporins, biofuels, butanol, ethanol, lactic acid, and itaconic acid, comprising:
a) fermenting a medium containing a source of xylose with a cell as defined in claim 1.

23. The process of claim 22, further comprising:
b) recovery of the fermentation product

24. The eukaryotic cells of claim 8 wherein said cells are used for fermentation of biomass from a xylose-carbon source containing media.

25. The eukaryotic cells of claim 8 wherein said cells are used as a biocatalyst in situ or in purified form for the production of isomerized sugar products or intermediates, preferably for isomerized sugar products.
